(19) United States
(12) Patent Application Publication
(10) Pub. No.: US 2009/0314060 A1
(43) Pub. Date: Dec. 24, 2009

(54) CIRCUIT ASSEMBLY FOR OPERATING A GAS SENSOR ARRAY

(75) Inventors: Siegbert Steinlechner, Leonberg (DE); Bernd Schumann, Rutesheim (DE); Thorsten Ochs, Schwieberdingen (DE); Bernhard Kamp, Ludwigsburg (DE)

Correspondence Address: MERCHANT & GOULD PC, P.O. BOX 2903, MINNEAPOLIS, MN, 55402-0903, US

(73) Assignee: Robert Bosch GmbH, Stuttgart (DE)

(21) Appl. No.: 11/920,617

(22) PCT Filed: May 2, 2006
(86) PCT No.: PCT/EP2006/061969

§ 371 Date: Sep. 9, 2009

(30) Foreign Application Priority Data
May 19, 2005 (DE) 10 2005 023 184.5

(51) Int. Cl. G01N 33/00; G01N 27/12; H01L 29/02

(52) U.S. Cl. 73/31.06; 257/43; 257/E29.002

(57) ABSTRACT

The invention relates to a circuit assembly for operating a sensor array, in particular, a gas sensor array for detecting gases, which comprises at least one signal line. According to said invention, a signal line is divided into two parallel line branches with a sensor and a diode, preferably a Schottky diode, arranged in each of said two parallel line branches, whereby the two diodes have opposite electrical polarity. The use of different polarity diodes permits actuation of both sensors through only one signal line. It can be determined if the current flows through one or the other of both sensors by merely polarizing the electrical potential applied to the signal line appropriately.

The invention concerns a circuit assembly to operate a sensor array, particularly a gas sensor array for the detection of exhaust gases according to the preamble of claim 1.

So-called sensor arrays are commonly used for the detection of gases, especially exhaust gases in automotive technology. These sensor arrays are constructed from multiple non-selective exhaust gas sensors, whereby one or several gases can be selectively detected with these arrays by means of appropriate signal evaluation, for example by a neural network.

In most cases in these sensor arrays, resistive semiconductor sensors are used for detection, for example those which are tin dioxide-based. A problem in using such arrays is that the sensor must be individually contacted, which in turn requires a large number of contacts on the sensor for external input leads. This leads particularly in the case of applications targeted by the automotive industry in the future, in which ceramic substrates are especially deployed, to the additional problem that the contacts must have very small dimensions and, moreover, must be disposed very closely next to each other. One such contact arrangement reduces significantly among other things the vibration resistance of the sensors, so that these can not be deployed in the automotive industry.

It is, thus, desirable to supply a circuit arrangement to operate, respectively to provide the electrical contacting for, such arrays with which the number of required contacts can be reduced.

### ADVANTAGES OF THE INVENTION

The idea underlying the invention at hand seeks to reduce the number of electrical contacts at the affected sensor arrays by the use of diodes, preferably by the use of inherently known Schottky diodes as metallic semiconductor junctions.

The circuit arrangement according to the invention to operate a sensor array, which has at least one signal line, has the distinctive characteristic; whereby the signal line, of which there is at least one, is divided into at least two parallel line branches. In these parallel line branches, of which there are at least two, a sensor and a diode are disposed in each case, whereby the diodes, of which there are at least two, are respectively reverse-biased.

By means of the deployment of differently polarized diodes, it is possible to actuate at least two sensors by way of a single signal line. Merely by polarizing the electrical potential impressed on the signal line appropriately, it can be determined if the measurement current is flowing through the one or respectively the other sensor; whereby the diodes, which are in each case disposed in a reverse-biasing operation, block in each case the preponderant proportion of the current through the line branch of the non-selected sensor or in the ideal situation essentially block in each case the entire current through the non-selected sensor.

In a preferred form of embodiment, Schottky diodes are used, and these are directly disposed on a ceramic substrate. In so doing, the number of the external input leads can further be reduced; and additionally the contacting problems mentioned at the beginning of the application can be reduced or even prevented. It is to be noted that Schottky diodes have when compared to conventional diodes, which are based on PN-junctions (for example in doped silicon or germanium), the particular advantage of being able to be produced in a form resilient to high temperature and can be in comparison to their conventional counterparts easily attached to the ceramic substrates previously mentioned. Thus, the circuit arrangement according to the invention can be manufactured by means of conventional thick film technology and therefore cost effectively. This especially is true if semiconductive metal oxides are used according to an additional form of embodiment.

It must be emphasized that the invention at hand can not only be deployed to operate the previously described gas sensor arrays with the advantages already mentioned, but in principle also with other sensor arrays constructed from other types of sensors, for example with regard to the subsequently described sensor arrays consisting of resistive and even non-resistive sensors, provided that at least two sensors can be operated by way of a single electrical signal line.

### DRAWINGS

The invention is described in more detail below using examples of embodiment, which are referenced to the attached drawing. Additional attributes, characteristics and advantages arise from these examples of embodiment.

In the drawing, the following items are shown in detail:

FIG. 1 a schematic description of the circuit arrangement according to the invention;

FIG. 2a a circuit arrangement according to the invention with an ohmic contact at a Schottky diode according to a first form of embodiment using different metals;

FIG. 2b a circuit arrangement according to the invention with an ohmic contact at a Schottky diode according to a second form of embodiment using a gradient in the doped concentration, respectively using consecutive layers of different semiconductors;

FIG. 3a a circuit arrangement according to the invention with a combination of a Schottky diode and a gas sensitive resistive sensor according to a first form of embodiment, in which an ohmic contact using different metals is implemented;

FIG. 3b a circuit arrangement according to the invention with a combination of a Schottky diode and a gas sensitive resistive sensor according to a second form of embodiment, in which an ohmic contact using different semiconductors, respectively doped gradients, is implemented; and

FIG. 4a-d variations of the circuit arrangement according to the invention, in which in each case multiple gas sensitive sensors are connected by only one signal line.

### DESCRIPTION OF EXAMPLES OF EMBODIMENT

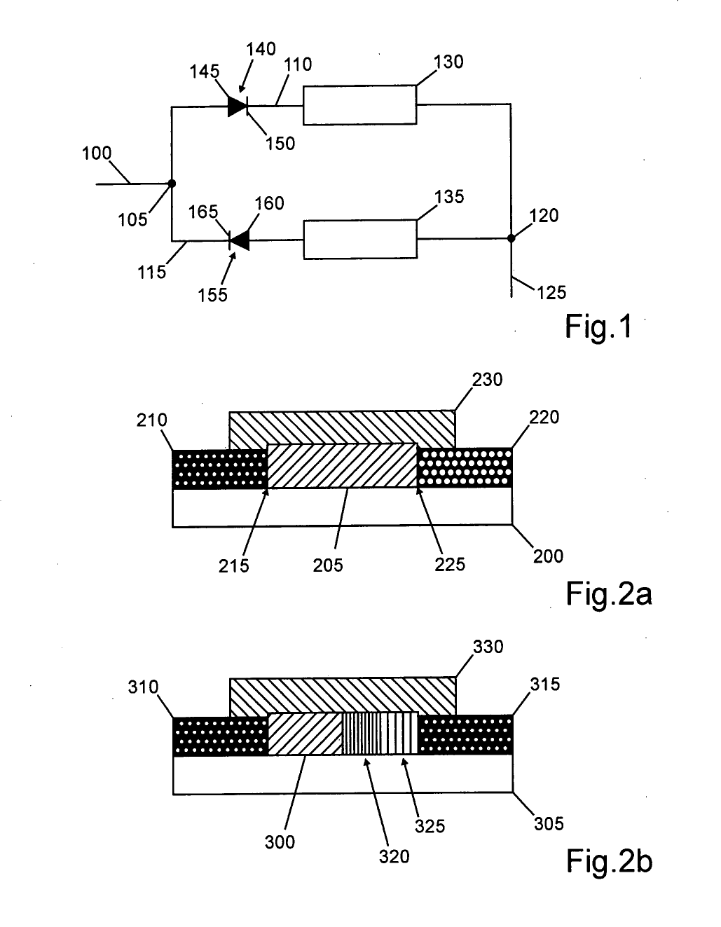

FIG. 1 shows a circuit arrangement according to the invention in a schematic description. A signal line 100 branches out at a first junction point 105 into two parallel line branches 110, 115. At a second junction point 120, the two line branches 110, 115 are brought together to form an outgoing dissipation line 125. In the examples of embodiment described below, a resistive sensor 130, 135 is disposed respectively in both line branches 110, 115, i.e. both sensors 130, 135 are operated by only the one signal line 100. It goes without saying that the invention at hand can also be deployed in principle using non-resistive sensors, provided these are also operated by way of an electrical signal line. A first Schottky diode 140 is disposed in the first line branch 110 with in fact its positive electrical pole pointing to the first junction point 105 and with its negative pole 150 pointing to the second junction point 120. A second Schottky diode 155 is disposed in the second line branch 115 and in fact in comparison to the first Schottky diode 155 with reversed polarity, i.e. with the positive pole 160 pointing to the first junction point 105 and the negative pole 165 pointing to the second junction point 120.

It is to be noted that it is presently not of concern, whether the Schottky diodes 140, 155 in the schematic representation are disposed to the left or to the right of the respective sensors 130, 135. It is also not of concern, how both of the Schottky diodes 140, 155 are electrically polarized. They must only have in each case reverse polarity.

By means of the polarity of one electrical potential impressed on the signal line 100, it can now be determined if the measurement current flowing through the signal line 100 and both of the line branches 110, 115 flows through the one sensor 130 or the other sensor 135. For that reason one of the two sensors 130, 135 can be selected merely by means of the polarization of the impressed potential. That is to say that first by the deployment of both of the Schottky diodes 140, 155 in the arrangement depicted in FIG. 1, it is possible to actuate the two resistive sensors 130, 135 by way of only the one signal line 100, respectively to select.

The Schottky diodes 140, 155 are preferably applied directly onto a ceramic substrate. In so doing, the number of external input leads can be additionally reduced as is subsequently described in detail. Moreover, in so doing the contacting problems mentioned at the beginning of the application are also reduced or even prevented. In this connection, the previously mentioned effect can be taken advantage of, in that the Schottky diodes can be manufactured in a form resilient to high temperatures. For that reason they can be easily applied onto ceramic substrates. Due to this fact, conventional thick film technology can be deployed. This is especially the case, if semiconductive metallic oxides are used.

As already mentioned at the beginning of the application, a Schottky diode consists of a metal-semiconductor-junction. The metal has a greater tendency to accept electrons than the semiconductor. For that reason, electrons leave an outer layer of the semiconductor to enter the metal. This layer with a reduced number of electrons acts as an obstruction to the current flow. Depending on the direction of an impressed potential, the effect of the obstructive outer layer can be increased or decreased.

Two of such metal-semiconductor-junctions lie inevitably along the route of the measurement current across the signal line 100, the respective selected sensor 130, 135 and the outgoing dissipation line 125. This is the case because the signal line 100 as well as the outgoing dissipation line 125 is likewise formed from a metal. This would lead to two diodes of opposed conducting directions being connected in series without any specific steps. The flow of current would therefore be blocked independently of the polarity of the measurement voltage. For that reason, it is required in most cases for both of the metallic semiconductor junctions to differentiate themselves to such a degree from each other that if possible only one of the two junctions creates an effect blocking the electrical current and the other one only acts as an ohmic contact.

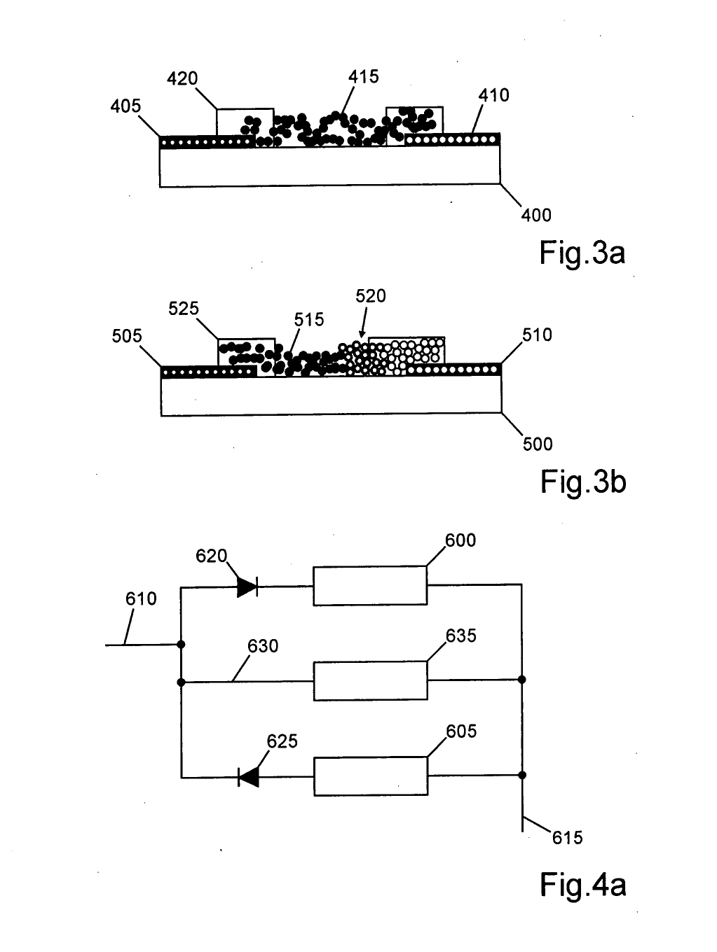

Schottky diodes of the existing type can be applied to a substrate having a gas sensor by different means. This is illustrated subsequently using the depicted examples of embodiment illustrated in FIGS. 2a and 2b. In both forms of embodiment depicted in FIGS. 2a and 2b, the Schottky diode is disposed separated from the actual gas sensitive sensor, whereas in both of the forms of embodiment depicted in FIGS. 3a and 3b, the Schottky diode is combined with the sensor, i.e. the sensor is integrated into the semiconductor of the Schottky diode.

The form of embodiment depicted in FIG. 2a comprises a substrate 200, upon which in the depiction at hand a semiconductor is applied in the middle. On the left side of the depiction, the semiconductor material 205 borders on a first input lead 210 made from a metallic conducting material with a relatively high electrical work function for electrons. At the interface 215 between the semiconductor 205 and the first metallic conductor 210, a first metal-semiconductor-junction, which deploys a blocking effect to the electrical current, forms itself in an inherently known manner. On the right side of the depiction at hand, the semiconductor material 205 borders on a second input lead 220 (respectively ‘outgoing dissipation line’ according to FIG. 1) made from a metallic conducting material, which has with regard to the first conducting material a relatively slight electrical work function for electrons. At the interface 225 between the semiconductor 205 and the second metallic conductor 220, a second metal-semiconductor-junction forms itself in an inherently known manner, which, however, acts only as an ohmic contact. The electronic characteristics previously mentioned of the first and the second metal-semiconductor-junctions serve to avoid the disadvantageous effect induced by the two metal-semiconductor-junctions, which has already been mentioned.

Because the composition of the gaseous ambiance can have an effect on the characteristics of the Schottky diodes, provision can be made for a protective surface, which separates the Schottky diode from the surrounding gaseous ambiance. Also, if the gas sensitive material acts itself as a semiconductor of the Schottky diode, provision can be made for the necessary protection between the metal and the semiconductor by covering the contact area. Provision is, therefore, made on the semiconductor layer 205 in the example of embodiment at hand for a top layer 230 to protect against such a gas effect. This top layer 230 completely covers the semiconductor 205 and extends in an overlapping fashion up to the areas of both of the input leads 210, 220.

As material for the semiconductor layer 205, high temperature resistant silicon carbide or semiconductive metal oxides (for example TiO₂, SnO₂, WO₃, Cr₂O₃) in diverse dopings come, for example, into consideration. As material for the metallic conductors, precious metals as, for example, gold, platinum, palladium, rhodium, respectively or alloys of these metals come into consideration. However, an application of metallic conductive oxides as, for example, lanthanum manganate, lanthanum chromite, lanthanum cobaltate is conceivable.

In the form of embodiment depicted in FIG. 2b, a semiconductor material 100 is once again applied in the center of a substrate 305. On the left side of the figure at hand, the semiconductor borders again on a first input lead 310 made from a metallic conducting material. On the right side of the figure at hand, an input, respectively outgoing dissipation, line is again disposed. Unlike FIG. 2a the semiconductor 300 in the area 320, 325 close to the second input lead 315 is doped for the reasons already mentioned, and in fact with a gradient in the doped concentration. The two partial areas 320, 325 represent in the example of embodiment at hand areas with a different degree of doping, i.e. the named gradient is achieved in reality by the discrete graduation of the degree of doping. In this form of embodiment the metals used for the contacting can be identical; respectively they approximately have the same work function.

Alternatively to the aforementioned doping gradient, provision can be made to dispose additional semiconductors in consecutive layers, whereby the layers likewise form preferably a gradient in the doping and in fact in the direction of the layering sequence. As in the example of embodiment according to FIG. 2a, provision can also here additionally be made for a top (protective) layer 330 with the aforementioned characteristics.

Subsequently the different implementation possibilities of the required ohmic contact, which have already been mentioned, will be explained, and in fact done so using conductive metal oxides. One such ohmic contact can in this case be produced in the following alternative ways:
1) The semiconductor is contacted with two different metallic conductors as depicted in FIG. 2a. The metal with the smaller tendency to accept electrons from the semiconductor forms the ohmic contact.
2) The semiconductor located between both of the metallic contacts is modified at the point of ohmic contact in such a way that its tendency to give off electrons to the metal is reduced. For this to occur, the following steps are, for example conceivable.
   a) The semiconductor is transferred at the point of the ohmic contact by means of a suitable doping from the semiconductive to the metallic (respectively band conductive) state (see FIG. 2b). In so doing, it can be expedient to use a slowly increasing doping gradient;
   b) A transitional layer made from an additional semiconductor material or several consecutive layers made from additional semiconductor materials is to be used. These layers have a tendency to progressively reduce the electrons given off to the metal.
3) The semiconductor is doped at the point of ohmic contact to such a degree that its charge carrier concentration will increase to such an extent that the thickness of the depletion edge layer reduces. In so doing, it can be expedient to use a slowly increasing doping gradient;
4) Optional combinations between the alternatives 1)-3) are possible.

It is to be noted that the alternatives 1) and 3) concern themselves with known technical procedures of the ohmic contacting of Schottky diodes based upon conventional semiconductors, such as Si or Ge.

In the additional forms of embodiment according to the FIGS. 3a and 3b, the respective gas sensitive material (semiconductive metal oxide, for example TiO₂, SnO₂, WO₃, Cr₂O₃) is used itself for the Schottky diode.

In the example of embodiment shown in FIG. 3a, a first input lead 405 made from a metallic conductor material with a relatively high electronic work function is disposed on a substrate 400 on the one (left in the drawing at hand) side. On the opposite (right in the drawing at hand) side, a second input lead respectively outgoing dissipation line, 410 is located, which is manufactured from a conductor material with a relatively small work function for electrons. A gas sensitive layer 415 made from semiconductive metal oxide is disposed between these two leads 405, 410—unlike the FIGS. 2a and 2b. As suggested by the particles, this layer 415 in the example of embodiment at hand is manufactured by means of thick film, respectively thick layer, technology. In the border areas of this gas sensitive layer 415, provision can be made likewise for a protective layer 420 against gas exposure.

In the example of embodiment depicted in FIG. 3b, input leads 505, 510 formed bilaterally from metallic conductors are disposed on a substrate 500. A gas sensitive layer 515 made from semiconductive metal oxide is again disposed between these two leads. On the depicted right side of the gas sensitive layer 515, provision is made, however, in the example of embodiment at hand for a gradient 520 in the doping concentration of the semiconductive metal oxide. In the border areas of the gas sensitive layer 515, provision can likewise be made for a protective layer 525 against gas exposure for the reasons which have already been mentioned.

It can be found in most of the applications that a voltage drop at the diode interferes with the measurement of resistance. For that reason, provision can be made according to an example of embodiment, which is graphically not depicted here, to not measure the resistance of the gas sensitive sensor with a direct-current voltage but with an alternating-current voltage, which is impressed on a constant bias voltage. By measuring the proportion of alternating current of the total current flowing through the sensor, it is possible to only selectively measure the resistance of the gas sensitive layer. By means of the polarization of the bias voltage, control is possible, as shown above, over which gas sensitive sensor is actuated. It is additionally possible during a direct-current measurement to use different voltage values (at least 2), which in each case are greater than the breakdown voltage of the Schottky diode. The resistance of the gas sensitive sensor results in an inherently known manner from the calculation of the slope of the respective current/voltage characteristic curve.

According to a form of embodiment, which is likewise not depicted here, one of the two Schottky diodes is dispensed with per signal line. In this case, only the resistance of a gas sensitive sensor is measured in the direction of current flow, in which the Schottky diode blocks. In the other direction of current flow, a summation signal is measured, which comes from both gas sensitive sensors.

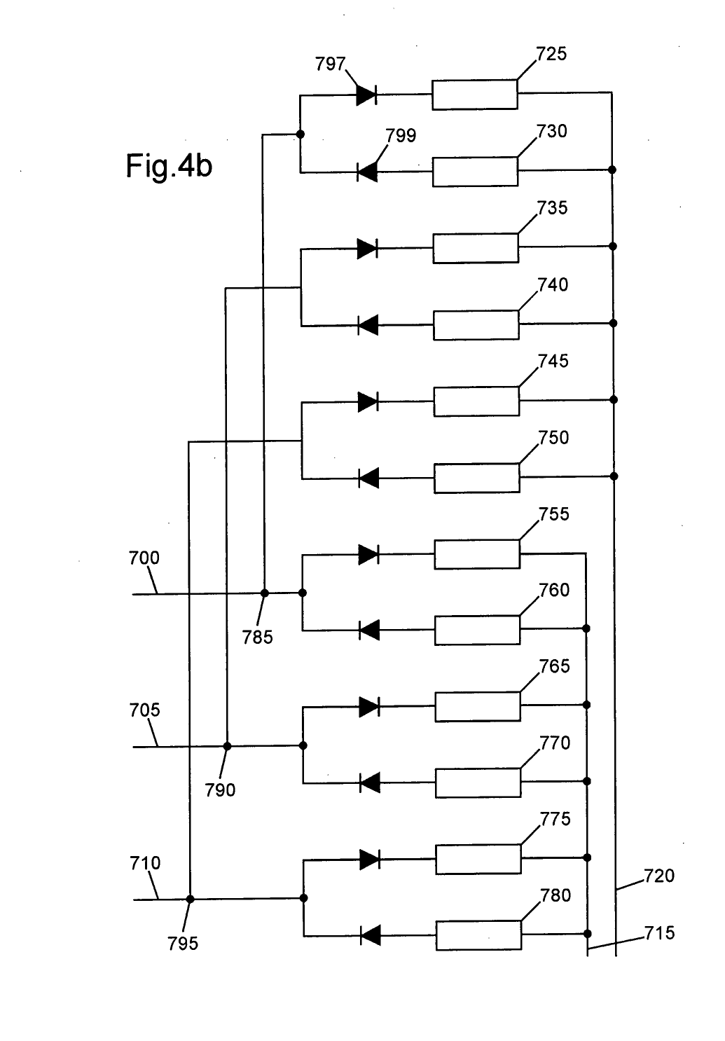

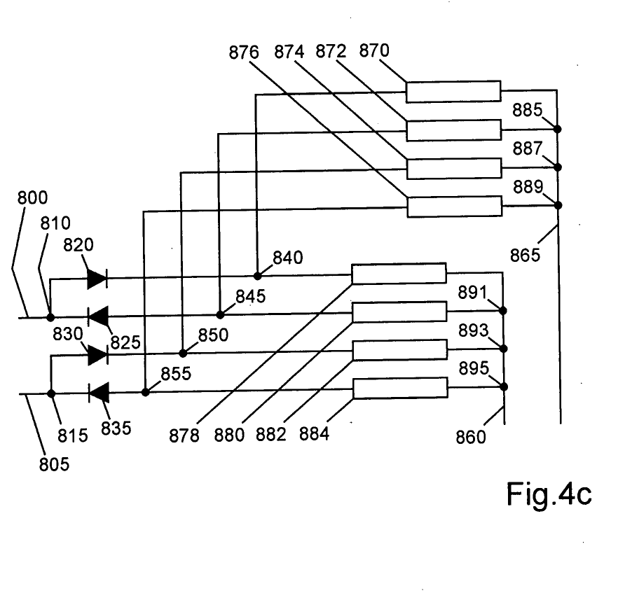

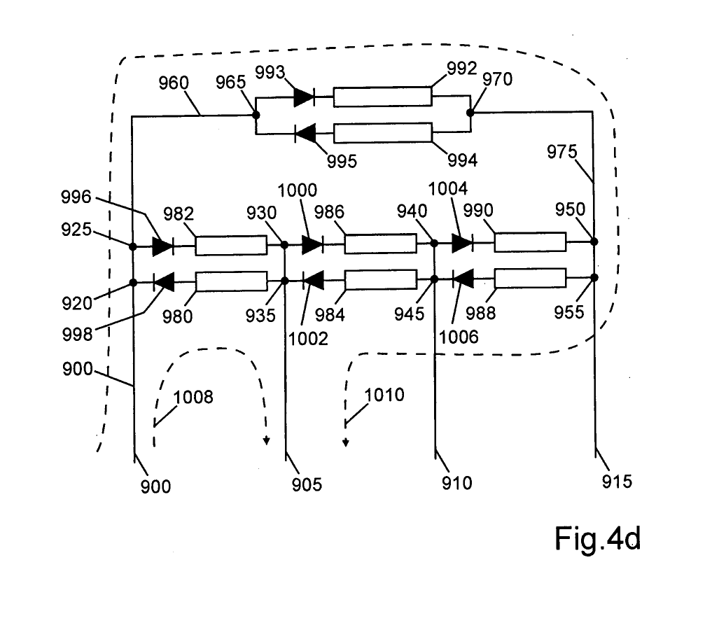

In the FIGS. 4a to 4d, different circuit variations are now shown for the operation, respectively formation, of one of the sensor arrays, which is of concern here. According to a first variation, three gas sensitive sensors are operated, respectively calibrated, by way of a signal line (see FIG. 4a). The circuit arrangement depicted in FIG. 4a has corresponding to FIG. 1 two resistive sensors 600, 605, which by means of a parallel circuit are operated by way of the one signal line 610 and the one outgoing dissipation line 615. These sensors 600, 605 are selected in the manner described by means of the two Schottky diodes 620, 625. The circuit arrangement comprises an additional parallel circuit loop 630, in which an additional resistive sensor is disposed. This parallel circuit loop 630 does not contain, however, a Schottky diode. In this variation when small measurement voltages are applied, only the resistance of sensor 635, which is not connected in series to the Schottky diode, is measured. In the case of voltages (positive or negative), which are greater than the breakdown voltage of the Schottky diodes 620, 625, a summation signal is once again measured. This first circuit variation is especially well suited, if the gas sensitive sensor 635, which is not coupled with a Schottky diode, has a significantly greater ohmic resistance than the sensors 600, 605, which are coupled with a Schottky diode. In this case, the gas sensitive sensor 635 not coupled with a Schottky diode interferes only slightly with the measurement of resistance of the other sensors 600, 605. However, this variation leads to a reduced accuracy in measurement.

As can be seen from the FIGS. 4b to 4d, the additional circuit variations have combinations from the previously described circuit variations, which in each case consist of Schottky diodes and gas sensitive resistive sensors in order to provide as high a number as possible of individual sensors in the sensor arrays. In the circuit depicted in FIG. 4b, a total number of 2*n*k individual sensors 725-780 is implemented with k signal lines 700-710 and n outgoing dissipation lines. The signal lines 700-710 separate themselves at the junction points 785-795 in each case into two parallel sensor pairs according to FIG. 1. In each case, two individual sensors 725, 730 etc. are assigned according to FIG. 1 to two Schottky diodes 797, 799 etc.

The variation depicted in FIG. 4c comprises k signal lines 800, 805, which separate themselves at two initial junction points 810, 815 (i.e. in the Figure at hand k=2) into in each case two parallel conductor pathways, in which respectively a Schottky diode 820-835 is disposed. At four second junction points 840-855, which are disposed with regard to the signal flow direction behind the Schottky diodes 820-835, the four parallel conductors separate themselves into 2×4 parallel conductor lines as depicted in the Figure at hand. An individual sensor 870-884 is disposed in each of these conductor lines. The 2×4 parallel conductor lines are brought together into two outgoing dissipation lines 860, 865 (as shown n=2) at six third connection points 885-895 as depicted in FIG. 4c. It goes without saying that the number of the signal lines 800, 805 and the outgoing dissipation lines 860, 865, i.e. the values from k and n, are only given priority; and for that reason, the sensor arrays can vary depending upon the purpose of the application, provided that the circuit requirements described in this application are fulfilled. The circuit variation at hand has the advantage of being able to save Schottky diodes; however, this is only feasible if the Schottky diodes can be assembled separated from the gas sensitive sensors. This is the case in the form of embodiment at hand because the second junction points 840-855 must be disposed between the Schottky diodes 820-835 and the sensors 870-884.

The variation depicted in FIG. 4d has n=4 signal lines 900-915. The signal lines 900-915 come together at the first junction points 920-955. By way of these first junction points 920-955, parallel conductor pathways are formed in each case, in which respectively a Schottky diode/sensor pair 996-1006, respectively 980-990, is disposed. An additional parallel conductor pathway 960, 975 is formed at both of the first junction points 925, 950. In the conductor pathway 960, 975, two additional parallel conductor pathways are formed at the second junction, respectively connection, points 965, 970. Within these two pathways two additional optional sensors 992, 994 with in each case an accompanying Schottky diode 993, 995 are disposed. By means of the dotted line 1008, a possible current measurement pathway (as indicated in FIG. 4d between the two signal lines 900, 905) is denoted, which is formed without any additional steps (i.e. automatically) by the corresponding polarity of the respective signal voltage as a result of the existing arrangement and polarity of the Schottky diodes 996-1006 and 993, 994. The dotted line 1010 denotes additionally in this current measurement pathway a possible pathway for leakage current 1010.

The variation depicted in FIG. 4d allows for an arrangement of 2*(n−1) individual sensors with n signal lines and, in fact, without the use of the optional gas sensitive sensors according to FIG. 4a. Taking into account the optional, additional, gas sensitive sensors, a sensor array of in total 2*n individual sensors is even made possible. When using the optional, additional sensors, the disadvantage previously mentioned, however, occurs; in that next to the actual measurement current, an additional leakage current can flow, which endangers the accuracy of the measurement. This leakage current can, however, be held to a minimum if the measurement voltage is indeed greater than the breakdown voltage of the Schottky diodes located along the current measurement pathway. This same measurement voltage must, however, stay smaller than the sum of the breakdown voltages of the Schottky diodes located along the pathway of the leakage current.

It must be emphasized that the invention can also be deployed with gas sensors, which are based on gas sensitive Schottky diodes instead of the resistive (layered) sensors. In this case, the assembly of an individual sensor corresponds to the assembly depicted in the FIGS. 3a and 3b. In this form of embodiment, however, at least a part of the aforementioned protective layer 420, 525 is omitted; and, in fact, the part, which is disposed above the contact 215, 225. This particular part unfolds the diode's effect, the aforementioned protective layer. The protective layer above the ohmic contact can, however, be maintained. Unlike the variations discussed above using resistive gas sensors, the resistance of the actual semiconductor layer is in this instance negligible. As a measurement signal in this example of embodiment, a necessary voltage for the constant flow of current through the Schottky diode is sensed.

1. A circuit arrangement for the operation of a gas sensor array that detects gases, the circuit arrangement comprising:
a sensor array including at least one signal line, where the at least one signal line, is divided into a first parallel line branch having at least a first sensor and a first diode connected in series, and a second parallel line branch having at least a second sensor and a second diode connected in series, whereby the first and second diodes are electrically reverse-biased, are formed by Schottky diodes, and are directly disposed on a ceramic substrate; whereby by means of a polarization of an electrical potential impressed on the signal line it is determined if at least a predominant proportion of electrical current flowing through the signal line flows through the first sensor of the first parallel line branch or through the second sensor of the second parallel line branch.

2. A circuit arrangement according to claim 1, wherein the first and second diodes are manufactured using a thick film technology.

3. A circuit arrangement according to claim 1, wherein the first and second sensors are formed from a semiconductor metal oxide.

4. A circuit arrangement according to claim 1, further comprising at least two metal-semiconductor junctions, wherein one of the at least two metal-semiconductor-junctions is designed to have a blocking effect on the electrical current and the respective other metal-semiconductor-junction is designed as an ohmic contact.

5. A circuit arrangement according to claim 1, wherein at least one of the first or second sensors is integrated into the respective semiconductor of the first or second diode.

6. A circuit arrangement according to claim 1, further comprising a protective layer disposed, which separates the diodes and/or the metal-semiconductor-junctions from a gaseous ambiance surrounding the sensor array.

7. A circuit arrangement according to claim 3, wherein the semiconductor metal oxide is formed from silicon carbide resistant to high temperature or from a semiconductive metal oxide, preferably TiO₂, SnO₃, WO₃, Cr₂O₃ in equal or differing dopings; and in that the signal line is formed from a precious metal, preferably gold, platinum, palladium, rhodium or alloys of these metals or from a metallic conductive oxide, preferably lanthanum manganate and/or lanthanum chromite and/or lanthanum cobaltate.

8. A circuit arrangement according to claim 3, wherein at least one of the first or second diodes an area of the signal line is doped with a gradient in the doping concentration or with a discrete graduation of the degree of doping.

9. (canceled)

10. A circuit arrangement according to claim 1, wherein an electrical resistance of at least one of the first or second sensors is measured with an alternating-current voltage, which is impressed on a constant bias voltage, whereby the electrical resistance of at least one of the first or second sensors is selectively sensed by means of a measurement of the proportion of alternating current of the total current flowing through the sensor; and whereby by means of the polarization of the bias voltage, control is taken over which sensor is actuated.

11. A circuit arrangement according to claim 1, wherein an electrical resistance of at least one of the first or second sensors is measured with direct-current voltage, whereby at least two differing voltage values are used, which in each case are greater than a breakdown voltage of the diode.

12. A circuit arrangement according to claim 1, wherein the at least one signal line, is divided into at least three parallel line branches, whereby in each of at least two of the at least three parallel line branches, at least one resistive sensor and at least one diode connected in series to the respective resistive sensor are disposed; and in that in the at least third line branch, an additional resistive sensor without a diode connected in series is disposed; whereby when relatively small measurement voltages are applied, only the resistance of the additional sensor is measured; and whereby in the case of voltages, which are greater than a breakdown voltage of the diodes, of which there are at least two, a summation signal is measured.
